(19) United States
(12) Patent Application Publication
(10) Pub. No.: US 2008/0021360 A1
(43) Pub. Date: Jan. 24, 2008

(54) ROLL-UP WOUND PROTECTOR

(76) Inventors: Carrie I. Fihe, Cincinnati, OH (US); Andrew T. Beckman, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/458,329

(22) Filed: Jul. 18, 2006

(51) Int. Cl. A61F 13/00

(52) U.S. Cl. 602/60

(57) ABSTRACT

A roll-up wound protector has a distal ring, a proximal ring, and a flexible sleeve extending between the proximal and distal rings. The proximal ring may have varying geometries and is rollable to gather the flexible sleeve around the proximal ring and shorten the length of the flexible sleeve.

### BACKGROUND

The present invention relates in general to surgical devices and procedures, and more particularly to wound protectors and wound retractors.

Surgical procedures are often used to treat and cure a wide range of diseases, conditions, and injuries. Many surgical procedures require access to internal tissue through open surgical procedures or endoscopic surgical procedures. The term “endoscopic” refers to all types of minimally invasive surgical procedures including laparoscopic and arthroscopic procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Trocars are often used to provide a port through which endoscopic surgical instruments are passed. Trocars generally have a sealing valve that prevent the insufflatory fluid from escaping while an instrument is positioned in the trocar. Sometimes hand access devices are also used during endoscopic surgery, often referred to as hand assisted laparoscopic surgery (“HALS”). A HALS device will typically seal around a surgeon's hand or arm to prevent the insufflatory fluid from escaping while allowing the surgeon to manipulate tissue within the patient's body.

While wound protectors and wound retractors are known, no one has previously made or used a wound protector or wound retractor in accordance with the present invention.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are drawn to scale and like reference numerals identify the same elements.

FIG. 1 depicts an non-scaled schematic view of a roll-up wound protector;

FIG. 2 depicts a non-scaled schematic view of a roll-up wound protector deployed in an abdomen;

FIG. 3 depicts a cross-sectional view of a proximal ring;

FIG. 4 depicts a cross-sectional view of a proximal ring;

FIG. 5 depicts a cross-sectional view of a proximal ring;

FIG. 6 depicts a cross-sectional view of a proximal ring;

FIG. 7 depicts a cross-sectional view of a proximal ring; and

FIG. 8 depicts a cross-sectional view of a proximal ring;

### DETAILED DESCRIPTION

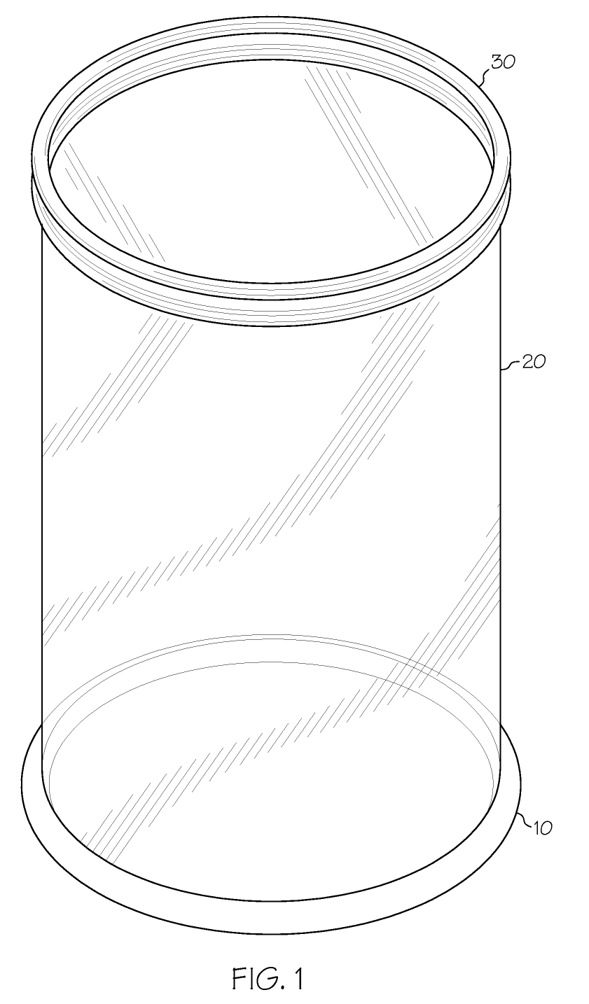

FIG. 1 depicts a perspective view of a roll-up wound protector in an extended position. The wound protector comprises a distal ring (10), a proximal ring (30), and a flexible sleeve (20) having a length extending between the proximal and distal rings. The wound protector can be used as a stand-alone device or in combination with a cap having sealing valve for endoscopic instruments or a surgeon's arm.

In this embodiment the distal ring (10) is circular with a circular cross-sectional geometry; however, non-circular rings and non-circular cross-sectional geometries are also possible. For instance, the distal ring could have a oval or elliptical in cross-sectional shape. The distal ring (10) can be made from a variety of different materials with different characteristics. In this example the distal ring is made from an elastomer such as polyurethane, polyethylene, silicone, and the like. The distal ring can also vary in size. For instance, the distal ring can have an inside diameter greater than 1 inches and less than 9 inches, and a thickness less than 1 inch, but dimensions outside these ranges are also possible. Optionally, the distal ring (10) will have a durometer between 40 A and 90 A or 70 D, but other material properties are also possible.

In this embodiment the sleeve (20) is a single layered tube of material; however, a discontinuous sleeve or multi-layered sleeves are also possible. The sleeve (20) can be made from a variety of variety of different materials with different characteristics. In one example, the sleeve (20) is made from an elastomer such as polyisoprene, silicone, polyurethane, silicone, and the like; however, inelastic materials such as mylar could also be used. The sleeve (20) may be clear, transparent, translucent, or opaque. As shown here, the sleeve (20) is fastened at its ends directly to the proximal and distal rings using an adhesive or heat sealing techniques; however, alternative techniques may also be employed. The sleeve (20) could also be attached to the rings at locations other than the sleeve ends. For instance, the sleeve (20) can wrapped around the distal ring (10) and adhesively attached or sealed to itself. The length of the sleeve (20) can also vary. For instance, the sleeve may be between 2 cm and 20 cm in length; however, other lengths are also possible. The thickness of the sleeve (20) can also vary. For instance, the sleeve thickness in this embodiment is between 0.010 and 0.020 inches; however, other thicknesses are also possible.

In this embodiment the proximal ring (30) is circular; however, non-circular rings are also possible. The proximal ring (30) can also vary in size. For instance, the proximal ring (30) can have an inside diameter between 1 and 9 inches, but other dimensions are also possible. Optionally, the ratio of the distal ring (10) and proximal ring (30) diameters is greater than 0.4. The proximal ring (30) can take a variety of different cross-sectional geometries. In this embodiment, the cross-sectional geometry has a height greater than the width and is substantially constant around the circumference of the proximal ring (30). A geometry is substantially constant if any variations are insignificant. For example, geometric variations resulting from molding or other manufacturing factors would be considered substantially constant. Also in this embodiment the cross-sectional geometry is substantially solid; however, holes or cavities may also be present.

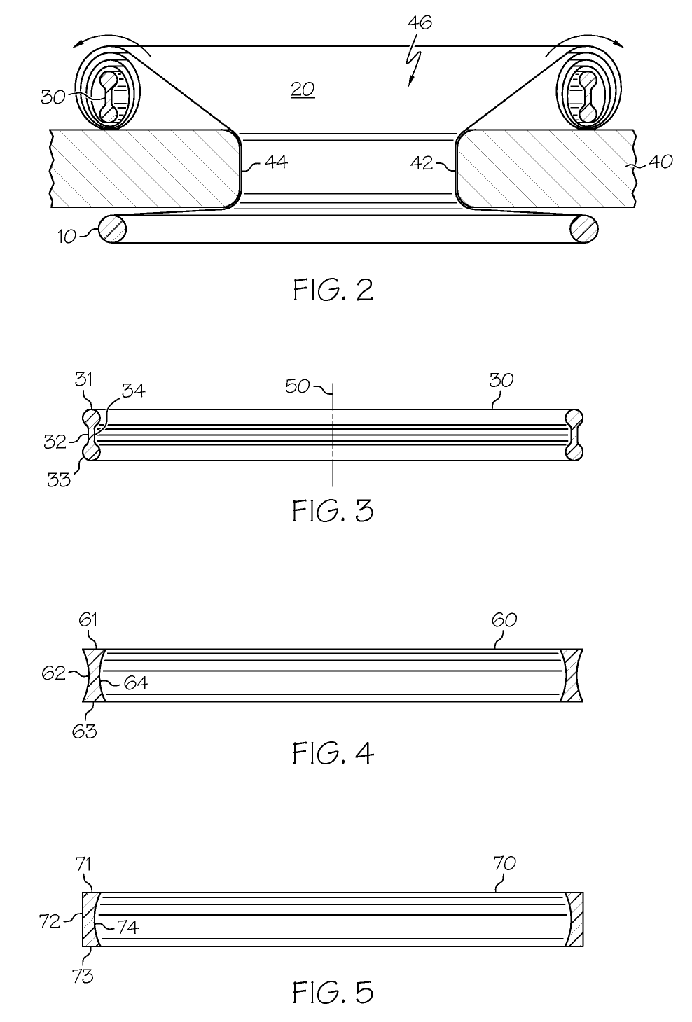

FIG. 2 depicts an example of the wound protector in a deployed position in a patient. In this example the wound protector in positioned in a patient's abdominal wall (40) through an incision (46). The distal ring (10) is held in a collapsed position (e.g., in an oblong shape like an oval, a peanut, a figure eight, and the like) to reduce its size and then inserted through the incision (46). After insertion, the distal ring (10) is released and then expands to its ring-like shape. As shown here, the expanded distal ring (10) is larger than the incision (46) and sits against the peritoneal surface of the abdominal wall (40). The proximal ring (30) is rollable to gather the flexible sleeve (20) around the proximal ring (30), and the wound proximal ring (30) sits on the cutaneous surface of the abdominal wall (40). The proximal ring (30) is rollable in the outward directions (as shown by the arrows) to shorten the sleeve (20) and in the inward direction to lengthen the sleeve (20), or vice versa. The shortening of the sleeve (20) pulls the sleeve (20) taut against the incised wound (42, 44). As one with ordinary skill in the art will recognize, surgical procedures can be performed through the incision (46) and the sleeve (20) protects the incised wound (42,44) from infection and contamination. In addition, the taut sleeve (20) tends to pull the incised wound (42,44) open thus functioning as a wound retractor. As demonstrated in this example, more retraction is possible by rolling the proximal ring (30) outward, while less retraction is possible by rolling the proximal ring (30) inward.

In this example the proximal ring (30) rolls in resting increments of 180 degrees. In other words, when the ring rolls it “snaps” between resting positions. Optionally, the flip force for the proximal ring (30) can be less 10 in*lbs/180 degrees of rotation, and can be less than 3 in*lbs. Flip force is a way of measuring the force required to roll the ring about itself. The flip force is measured at room temperature on a stand-alone proximal ring without the sleeve attached. An equal and opposite torque is applied simultaneously to a ring at two diametrically opposite points along the circumference of the ring. The peak measured torque to roll the ring is used to calculate the flip force. By compiling 100 peak measured torques for a given ring, the statistical median value is the flip force. Preferably, the flip force is substantially the same for each sequential resting incremental rotation. Optionally, the proximal ring (30) may have substantially no residual hoop stress. One way to achieve this is through a molding process where the proximal ring (30) is injection molded and transfer molded using a thermoplastic or thermoset elastomer such as polyisoprene, silicone, polyurethane, silicone, and the like. In one embodiment, the proximal ring is molded from Desmopan 9370. The proximal ring (30) may have a durometer between 50 A and 50 D, but other material properties are also possible.

FIGS. 3 illustrates an example of a cross-sectional geometry of the proximal ring (30). The proximal ring (30) comprises a central axis (50). The cross-sectional shape shown in this example is solid and comprises a bulbous proximal portion (31), shown here as a generally circular shape, a bulbous distal portion (33), also shown here a generally circular shape. A connecting member having a lateral flat surface (32) and a medial flat surface (64) is interposed between and connected to the bulbous portions (31, 33). The lateral and medial surfaces (32, 34) are parallel one another and parallel the central axis (50). In this embodiment the bulbous portions (31, 33) are symmetrical one another, and the lateral and medial surfaces (32, 34) are symmetrical one another. The surfaces are substantially constant about the circumference of the proximal ring (30). In the present embodiment the various surfaces transition smoothly, but geometric abrupt transitions are also possible.

FIG. 4 illustrates another example of a cross-sectional geometry of a proximal ring (60). The cross-sectional geometry is solid and includes a flat proximal surface (61), a concave arcuate lateral surface (62), a flat distal surface (63), and a concave arcuate medial surface (64). The proximal and distal surfaces (61, 63) are parallel one another and normal the central axis. The proximal and distal surfaces (61, 63) are symmetrical one another, and the lateral and medial surfaces (62, 64) are symmetrical one another. In the present embodiment the various surfaces have relatively abrupt geometric transitions. The cross-sectional geometry has a height greater than the width and is substantially constant around the circumference of the proximal ring (60). The proximal ring (60) rolls in increments of 180 degrees. Optionally, the proximal ring (60) is made with a molding process and may have substantially no residual hoop stress.

FIG. 5 illustrates yet another example of a cross-sectional geometry of a proximal ring (70). The cross-sectional geometry is solid and includes a flat proximal surface (71), a flat lateral surface (72), a flat distal surface (73), and a concave arcuate medial surface (74). The proximal and distal surfaces (71, 73) are parallel one another and normal the central axis. The shapes of the lateral and medial surfaces (72, 74) may be reversed. In the present embodiment the various surfaces have relatively abrupt geometric transitions. The cross-sectional geometry has a height greater than the width and is substantially constant around the circumference of the proximal ring (70). The proximal ring (70) rolls in increments of 180 degrees. Optionally, the proximal ring (70) is made with a molding process and may have substantially no residual hoop stress.

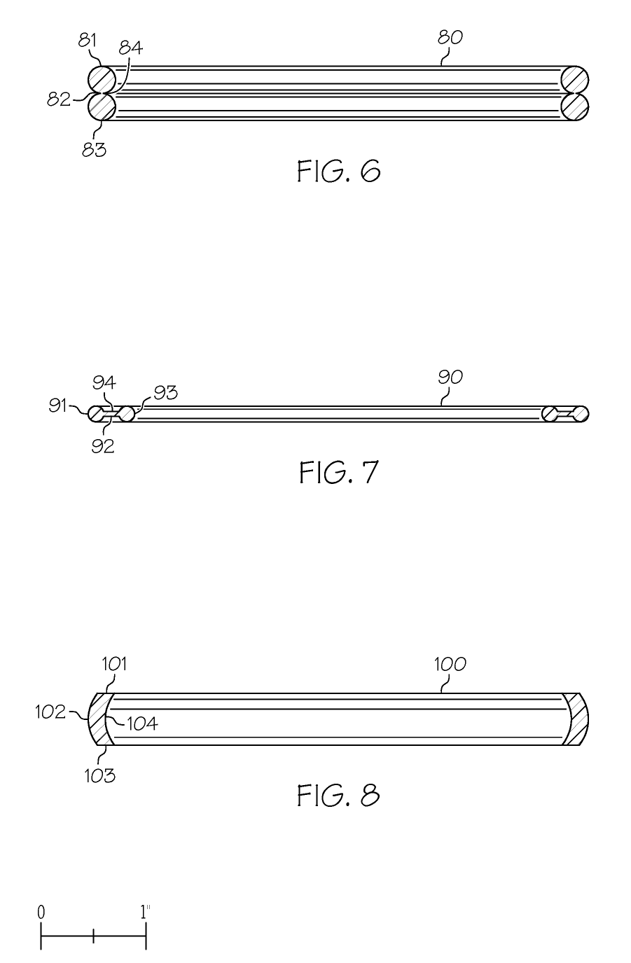

FIG. 6 illustrates still another example of a cross-sectional geometry of a proximal ring (80). The cross-sectional shape is solid and comprises a bulbous proximal portion (81), shown here as a generally circular shape, a bulbous distal portion (83), also shown here a generally circular shape. The junction of the two bulbous portions (82, 84) define a lateral surface (82) with a concave v-shape, and a medial surface (84) with a concave v-shape. The bulbous portions (81, 83) are symmetrical one another, and the lateral and medial surfaces (82, 84) are symmetrical one another. In the present embodiment the various surfaces have relatively abrupt geometric transitions. The cross-sectional geometry has a height greater than the width and is substantially constant around the circumference of the proximal ring (80). The proximal ring (80) rolls in increments of 180 degrees. Optionally, the proximal ring (80) is made with a molding process and may have substantially no residual hoop stress.

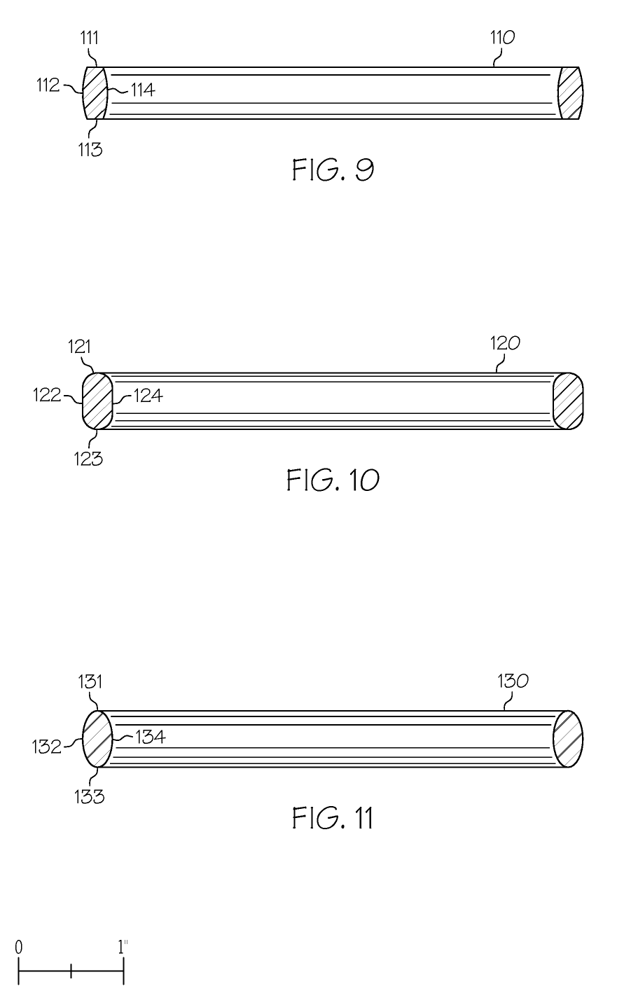

FIG. 7 illustrates another example of a cross-sectional geometry of a proximal ring (90). The cross-sectional shape is solid and comprises a bulbous lateral portion (91), shown here as a generally circular shape, a bulbous medial portion (93), also shown here a generally circular shape. A connecting member having a distal flat surface (92) and a proximal flat surface (94) is interposed between and connected to the bulbous portions (91, 93). The proximal and distal surfaces (92, 94) are parallel one another and normal the central axis. In this embodiment the bulbous portions (91, 93) are symmetrical one another, and distal and proximal surfaces (92, 94) are symmetrical one another. In the present embodiment the various surfaces transition smoothly, but geometric abrupt transitions are also possible. The cross-sectional geometry has a width greater than the height and is substantially constant around the circumference of the proximal ring (90). The proximal ring (90) rolls in increments of 180 degrees. Optionally, the proximal ring (90) is made with a molding process and may have substantially no residual hoop stress.

FIG. 8 illustrates another example of a cross-sectional geometry of a proximal ring (100). The cross-sectional geometry is solid and includes a flat proximal surface (101), a convex arcuate lateral surface (102), a flat distal surface (103), and a concave arcuate medial surface (104). The proximal and distal surfaces (101, 103) are parallel one another and normal the central axis. The shapes of the lateral and medial surfaces (102, 104) may be reversed. In the present embodiment the various surfaces have relatively abrupt geometric transitions. The cross-sectional geometry has a height greater than the width and is substantially constant around the circumference of the proximal ring (100). The proximal ring (100) rolls in increments of 180 degrees. Optionally, the proximal ring (100) is made with a molding process and may have substantially no residual hoop stress.

FIG. 9 illustrates another example of a cross-sectional geometry of a proximal ring (110). The cross-sectional geometry is solid and includes a flat proximal surface (111), a convex arcuate lateral surface (112), a flat distal surface (113), and a convex arcuate medial surface (114). The proximal and distal surfaces (111, 113) are parallel one another and normal the central axis. The lateral and medial surfaces (112, 114) are symmetric one another. In the present embodiment the various surfaces have relatively abrupt geometric transitions. The cross-sectional geometry has a height greater than the width and is substantially constant around the circumference of the proximal ring (110). The proximal ring (110) rolls in increments of 180 degrees. Optionally, the proximal ring (110) is made with a molding process and may have substantially no residual hoop stress.

FIG. 10 illustrates another example of a cross-sectional geometry of a proximal ring (120). The cross-sectional geometry is solid and approximates an oval, including a convex arcuate proximal surface (121), a flat lateral surface (122), a convex arcuate distal surface (123), and a flat medial surface (124). The lateral and medial surfaces (122, 124) are parallel one another and parallel the central axis. The proximal and distal surfaces (121, 123) are symmetrical one another. In the present embodiment the various surfaces transition smoothly. The cross-sectional geometry has a height greater than the width and is substantially constant around the circumference of the proximal ring (120). The proximal ring (120) rolls in increments of 180 degrees. Optionally, the proximal ring (120) is made with a molding process and may have substantially no residual hoop stress.

FIG. 11 illustrates another example of a cross-sectional geometry of a proximal ring (130). The cross-sectional geometry is solid and approximates an ellipse, including a convex arcuate proximal surface (131), a convex arcuate lateral surface (132), a convex arcuate distal surface (133), and a convex arcuate medial surface (134). The proximal and distal surfaces (121, 123) are symmetrical one another, and the lateral and medial surfaces (132, 134) are symmetrical one another. In the present embodiment the various surfaces transition smoothly. The cross-sectional geometry has a height greater than the width and is substantially constant around the circumference of the proximal ring (130). The proximal ring (130) rolls in increments of 180 degrees. Optionally, the proximal ring (130) is made with a molding process and may have substantially no residual hoop stress.

Preferably, the wound protectors described above will be processed before surgery. First, a new or used wound protector is obtained and if necessary cleaned. The wound protector can then be sterilized. In one sterilization technique the wound protector is placed in a closed and sealed container, such as a plastic or TYVEK bag. Optionally, the wound protector can be bundled in the container as a kit with other components, including one or more of the following: a sealing cap to maintain pneumoperitoneum, a sealing cap with a valve to allow passage of surgical instruments or a surgeon's arm while maintaining pneumoperitoneum (e.g., iris valve, gel seal, cuff, and the like), a tube of lubricant, a mounting ring in which the proximal ring may be seated and to which a cap can be attached, a marker, an incision template or scale, an instruction sheet, and the like. The container and wound protector, as well as any other components, are then placed in a field of radiation that can penetrate the container, such as gamma radiation, x-rays, or high-energy electrons. The radiation kills bacteria on the wound protector and in the container. The sterilized wound protector can then be stored in the sterile container. The sealed container keeps the wound protector sterile until it is opened in the medical facility.

The wound protectors described above can be used as a stand-alone device, for instance in open surgical procedures, or in combination with a cap having sealing valve for endoscopic instruments or a surgeon's arm. Among other advantages, the foregoing examples provide effective wound protection to prevent infection and facilitate wound retraction. Because the sleeve rolls-up, its length can be adjusted by the surgeon for any given anatomy and patient. Further, the surgeon can select the amount of retraction desired for a given procedure. The cross-sectional shapes of the proximal ring are easy to grip thus facilitating ease of use. Furthermore, the flip forces are relatively low and constant, further facilitating ease of use.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical wound protector, comprising:
a) a distal ring;
b) a proximal ring molded from an elastomer, the proximal ring having substantially no residual hoop stress; and
c) a flexible sleeve having a length extending between the proximal and distal rings;
wherein the proximal ring is rollable to gather the flexible sleeve around the proximal ring and shorten the length of the flexible sleeve.

2. The surgical wound protector of claim 1, wherein the proximal ring is solid.

3. The surgical wound protector of claim 1, wherein the shape of the proximal ring is substantially constant around the proximal ring.

4. The surgical wound protector of claim 1, wherein the proximal ring has a cross-sectional geometry with two bulbous portions and a flat portion interposed between and connected to the bulbous portions.

5. The surgical wound protector of claim 4, wherein the flat portion is parallel to the proximal ring central axis.

6. The surgical wound protector of claim 1, wherein the proximal ring has a cross-sectional geometry with a medially facing concave arcuate surface, a laterally facing concave arcuate surface symmetric to the medial surface, a proximally facing flat surface, and a distally facing flat surface parallel to the proximal surface.

7. The surgical wound protector of claim 1, wherein the proximal ring has a cross-sectional geometry with a medially facing concave arcuate surface and a laterally facing flat surface.

8. The surgical wound protector of claim 1, wherein the proximal ring has a cross-sectional geometry with a solid proximal circle connected to a solid distal circle, the proximal and distal circles being symmetric.

9. The surgical wound protector of claim 1, wherein the proximal ring has a cross-sectional geometry with a medially facing convex arcuate surface, a laterally facing convex arcuate surface symmetric to the medial surface, a proximally facing flat surface, and a distally facing flat surface parallel to the proximal surface, wherein the length of the arcuate surfaces is greater than the length of the flat surfaces.

10. The surgical wound protector of claim 1, wherein the proximal ring has a cross-sectional geometry comprising a longitudinally arranged solid oval or solid ellipse.

11. The surgical wound protector of claim 1, wherein the proximal ring has a cross-sectional geometry with a medially facing concave arcuate surface, a laterally facing convex arcuate surface, a proximally facing flat surface, and a distally facing flat surface parallel to the proximal surface, wherein the length of the arcuate surfaces is greater than the length of the flat surfaces.

12. The surgical wound protector of claim 1, wherein the sleeve is made from an elastomer.

13. A sterile surgical kit, comprising a sealed container containing therein the surgical wound protector of claim 1 and a sealing cap with a valve.

14. The surgical wound protector of claim 1, wherein the proximal ring rolls in resting increments of 180 degrees.

15. A method of processing a wound protector for surgery, comprising:
a) obtaining the surgical wound protector of claim 1;
b) sterilizing the wound protector; and
c) storing the wound protector in a sterile container.

16. A surgical wound protector, comprising:
a) a distal ring;
b) a proximal ring having a cross-sectional geometry with a solid proximal circle and a solid distal circle, the proximal and distal circles being symmetric; and
c) a flexible sleeve extending between the proximal and distal rings;
wherein the proximal ring is rollable to gather the flexible sleeve around the proximal ring.

17. The surgical wound protector of claim 16, wherein the cross-sectional geometry has a flat portion interposed between and connected to the proximal and distal circles.

18. The surgical wound protector of claim 16, wherein the proximal ring has substantially no residual hoop stress.

19. A sterile surgical kit, comprising a sealed container containing therein the surgical wound protector of claim 16 and a sealing cap with a valve.

20. A surgical wound protector, comprising:
a) a distal ring;
b) a proximal ring having a cross-sectional geometry with a two symmetric bulbous portions and a flat connecting portion interposed between and connected to the bulbous portions; and
c) a flexible sleeve extending between the proximal and distal rings.
